(19) United States
(12) Patent Application Publication
(10) Pub. No.: US 2023/0041980 A1
(43) Pub. Date: Feb. 9, 2023

(54) METHOD OF PROTECTING A RESPIRATORY TRACT OR A LUNG FROM DAMAGE, METHOD OF PROTECTING A LUNG FROM PRESSURE DAMAGE INDUCED BY A VENTILATOR, METHOD OF SUPPRESSING SPREAD OF INFECTION WITH VIRUSES OR BACTERIA THAT CAUSE PNEUMONIA IN PULMONARY BRONCHIAL EPITHELIAL CELLS OR ALVEOLAR EPITHELIAL CELLS, AND NASAL SPRAY

(71) Applicant: NEXT21 KABUSHIKI KAISHA, Tokyo (JP)

(72) Inventors: Shigeki SUZUKI, Tokyo (JP); Nobuo SASAKI, Tokyo (JP); Ungil CHUNG, Tokyo (JP); Nobuyuki SHIMOHATA, Kusatsu-shi (JP)

(73) Assignee: NEXT21 KABUSHIKI KAISHA, Tokyo (JP)

(21) Appl. No.: 17/967,775

(22) Filed: Oct. 17, 2022

Related U.S. Application Data
(63) Continuation-in-part of application No. PCT/JP2021/015605, filed on Apr. 15, 2021.

(30) Foreign Application Priority Data
Apr. 17, 2020 (JP) 2020-074281

(51) Int. Cl. A61K 31/7016; A61K 31/56; A61K 9/00; A61K 31/593; A61K 31/125; A61P 11/00

(52) U.S. Cl. CPC A61K 31/7016; A61K 31/56; A61K 9/0078; A61K 31/593; A61K 31/125; A61P 11/00

(57) ABSTRACT

A method of protecting a respiratory tract or a lung from damage is provided. The method comprises administering a pharmaceutical composition to a patient in need thereof. The pharmaceutical composition comprises trehalose or a trehalose derivative.

### CROSS-REFERENCE TO RELATED APPLICATIONS

This application is a continuation-in-part of International Patent Application No. PCT/W2021/015605 filed on Apr. 15, 2021, which claims priority to and the benefit of Japanese Patent Application No. 2020-074281 filed on Apr. 17, 2020, the entire disclosures of which are incorporated herein by reference.

### TECHNICAL FIELD

The present invention relates to a pharmaceutical composition containing trehalose or a trehalose derivative and a nasal spray, and particularly to a pharmaceutical composition for suppressing damage to the lung or respiratory tract.

### BACKGROUND ART

A large number of elderly persons die of, in particular, pulmonary tissue lesion caused by pneumonia or the like. For example, infectious pulmonary lesion is caused by a serious respiratory infection such as bacterial or viral pneumonia, aspiration pneumonia, or the like. Examples of bacteria that are known to cause pneumonia include pneumococcus and the like, and examples of viruses that cause pneumonia include influenza virus and the like. In recent years, pneumonia caused by novel coronaviruses (SARS, NIERS, and Covid-19) has become a major issue. Furthermore, various diseases such as serious infections including sepsis may occur as indirect damage associated with infectious pulmonary lesion.

Bacterial pneumonia generally occurs as “alveolar pneumonia” involving inflammation in “alveoli”, which are small sacs located at the end of the respiratory tract. In the case of this type of pneumonia, thick shadow is observed in a CT image. On the other hand, in the case of “interstitial pneumonia” involving inflammation in “interstitial tissues” located between alveoli and blood vessels, the interstitial tissues are hardened due to fibrosis, which may lead to dyspnea due to inability to take oxygen into the body through the lung. Regarding pneumonia caused by viral infection, interstitial pneumonia mainly occurs in many cases, and a serious condition thereof is diagnosed as acute interstitial pneumonia.

It is known that acute pulmonary lesion results from damage to pulmonary microvascular endothelial cells and alveolar epithelial cells caused by mediators such as tumor necrosis factor alpha (INFO that has been induced in an excessive amount in a living organism, rather than direct damage to pulmonary cells caused. by bacterial cell components or exotoxin. For example, an onset mechanism of Covid-19 is considered as follows: viruses proliferate in alveolar epithelial cells and infect alveolar macrophages while causing pulmonary damage, which results in local inflammation.

International Publication No. 2012/147705 discloses use of trehalose as a therapeutic agent for allergic diseases. The test results shown in International Publication No. 2012/147705 suggest that trehalose can reduce increased hypersensitivity of the respiratory tract involved in bronchial asthma and alleviate a symptom of asthma.

### SUMMARY OF INVENTION

According to an embodiment of the present invention, a method of protecting a respiratory tract or a lung from damage comprises administering a pharmaceutical composition to a patient in need thereof, wherein the pharmaceutical composition comprises trehalose or a trehalose derivative.

According to another embodiment of the present invention, a method of protecting a lung from oxygen comprises administering a pharmaceutical composition to a patient in need thereof, wherein the pharmaceutical composition comprises trehalose or a trehalose derivative.

According to still another embodiment of the present invention, a method of protecting a lung from dryness comprises administering a pharmaceutical composition to a patient in need thereof, wherein the pharmaceutical composition comprises trehalose or a trehalose derivative.

According to yet another embodiment of the present invention, a method of protecting a lung from pressure damage induced by a ventilator comprises administering a pharmaceutical composition to a patient in need thereof, wherein the pharmaceutical composition comprises trehalose or a trehalose derivative.

According to still yet another embodiment of the present invention, a method of suppressing propagation of cell death to surrounding cells induced by cell death comprises administering a pharmaceutical composition to a patient in need thereof, wherein the pharmaceutical composition comprises trehalose or a trehalose derivative.

According to yet still another embodiment of the present invention, a method of suppressing cell death comprises administering a pharmaceutical composition to a patient in need thereof, wherein the pharmaceutical composition comprises trehalose or a trehalose derivative.

According to still yet another embodiment of the present invention, a method of protecting pulmonary microvascular endothelial cells or alveolar epithelial cells comprises administering a pharmaceutical composition to a patient in need thereof, wherein the pharmaceutical composition comprises trehalose or a trehalose derivative.

According to yet still another embodiment of the present invention, a method of alleviating anoxic conditions in a lung by reducing airway resistance comprises administering a pharmaceutical composition to a patient in need thereof, wherein the pharmaceutical composition comprises trehalose or a trehalose derivative.

According to still yet another embodiment of the present invention, a method of suppressing infection with viruses or bacteria that cause pneumonia comprises administering a pharmaceutical composition to a patient in need thereof, wherein the pharmaceutical composition comprises trehalose or a trehalose derivative.

According to yet still another embodiment of the present invention, a method of suppressing spread of infection with viruses or bacteria that cause pneumonia in pulmonary bronchial epithelial cells or alveolar epithelial cells comprises administering a pharmaceutical composition to a patient in need thereof, wherein the pharmaceutical composition comprises trehalose or a trehalose derivative.

According to still yet another embodiment of the present invention, a method of moisturizing an airway or a lung comprises administering a pharmaceutical composition to a patient in need thereof, wherein the pharmaceutical composition comprises trehalose or a trehalose derivative. Since trehalose has a moisturizing effect, this pharmaceutical composition can prevent sputum from drying and moisturize an airway or a lung.

According to yet still another embodiment of the present invention, a solution pharmaceutical composition is administered intranasally with an administration device such as a spray or nebulizer, or is administered into an airway as a mist. The nasally administered solution passes from a nasal cavity to a posterior part of a pharynx, and together with substances attached to thereto, is swallowed into an esophagus and expelled into the stomach. Alternatively, the nasally administered solution is expelled from the nose along with substances attached to the nasal cavity. Alternatively, the solution administered into the airway will go to bronchi, bronchioles, and alveoli, and then will be carried to the pharynx along with substances attached thereto, and then will be swallowed into the esophagus and stomach, or will be expelled through an oral cavity to the exterior of the body as sputum.

According to still yet another embodiment of the present invention, a pharmaceutical composition is administered intravenously through a central vein. According to this embodiment, trehalose or a trehalose derivative can reach a blood vessel at alveoli via a pulmonary artery and diffuse into the alveoli. The pharmaceutical composition administered intravenously in the central vein passes through a vena cava, a right atrium, a right ventricle, and a pulmonary artery to an alveolar microvessel. The pharmaceutical composition further passes through a left atrium and a left ventricle to a coronary artery and a cerebral artery.

According to yet still another embodiment of the present invention, a nasal spray contains a pharmaceutical composition, wherein the pharmaceutical composition is a liquid formulation that is nasally administered using a spray or a nebulizer and contains trehalose or a trehalose derivative in an amount of 3.5 to 20 wt % and aqua solution.

Other features and advantages of the present invention will be apparent from the following description taken in conjunction with the accompanying drawings.

### BRIEF DESCRIPTION OF DRAWINGS

FIG. 1 The accompanying drawings, which are incorporated in and constitute a part of the specification, illustrate embodiments of the invention and, together with the description, serve to explain principles of the invention.

FIG. 1A shows the measurement results of the IgE antibody levels in Example 1.

FIG. 1B shows the measurement results of the IgE antibody levels in Example 1.

FIG. 2A shows the measurement results of airway resistance and compliance in Example 2.

FIG. 2B shows the measurement results of airway resistance and compliance in Example 2.

FIG. 3A shows the results of histological stain in Example 3.

FIG. 3B shows the results of histological stain in Example 3.

FIG. 4 shows the measurement results of the numbers of various cells in Example 4.

FIG. 5A shows the measurement results of the cytokine levels in Example 5.

FIG. 5B shows the measurement results of the cytokine levels in Example 5.

FIG. 5C shows the measurement results of the cytokine levels in Example 5.

FIG. 6A shows the measurement results after the application of hydrogen peroxide stimulus in Example 6.

FIG. 6B shows the measurement results after the application of hydrogen peroxide stimulus in Example 6.

FIG. 7A shows the measurement results of the cytokine levels in Example 7.

FIG. 7B shows the measurement results of the cytokine levels in Example 7.

FIG. 7C shows the measurement results of the cytokine levels in Example 7.

FIG. 7D shows the measurement results of the cytokine levels in Example 7.

FIG. 7E shows the measurement results of the cytokine levels in Example 7.

FIG. 8A shows the measurement results after the application of drying stimulus in Example 8.

FIG. 8B shows the measurement results after the application of drying stimulus in Example 8.

### DESCRIPTION OF EMBODIMENTS

Hereinafter, embodiments will be described in detail with reference to the attached drawings. Note, the following embodiments are not intended to limit the scope of the claimed invention, and limitation is not made an invention that requires a combination of all features described in the embodiments. Two or more of the multiple features described in the embodiments may be combined as appropriate.

Demand for a pharmaceutical composition that can reduce damage to the lung or respiratory tract as mentioned above, particularly a symptom of an infectious respiratory disease involving pulmonary lesion, has increased.

According to an embodiment of the present invention, it is possible to provide a pharmaceutical composition capable of suppressing damage to the lung or respiratory tract.

A pharmaceutical composition according to one embodiment of the present invention contains trehalose or a trehalose derivative. Trehalose is a known compound and is commercially available. Trehalose includes hydrated crystalline trehalose, anhydrous crystalline trehalose, and trehalose-containing carbohydrate solutions. Trehalose also includes α,α-trehalose (narrowly-defined trehalose), α,β-trehalose (neotrehalose), and β,β-trehalose (isotrehalose). In one embodiment, hydrated crystalline trehalose or anhydrous crystalline trehalose derived from α,α-trehalose (α-D-glucopyranosyl α-D-glycopyranoside) is used as the trehalose.

The trehalose derivative refers to a trehalose prodrug or a compound obtained by modifying a substituent of trehalose. The trehalose prodrug refers to a compound that produces trehalose before or after administered. For example, a compound that is obtained by providing a protecting group to trehalose and produces trehalose in a living organism after administered is a trehalose prodrug.

Examples of the compound obtained by modifying a substituent of trehalose include glycosylated derivatives of trehalose, acylated derivatives obtained through acylation, sulfated trehalose obtained through sulfation or pharmaceutically acceptable salts thereof, and phosphorylated trehalose obtained. through phosphorylation or pharmaceutically acceptable salts thereof.

Examples of the glycosylated derivatives of trehalose include irreducible oligosaccharides that include three or more glucoses and have a trehalose structure in the molecule. The glycosylated derivatives of trehalose may be carbohydrates having a trehalose structure at the terminus of the molecule. Examples of the carbohydrates having a trehalose structure at the terminus of the molecule include glucosyl trehalose such as α-glucosyl α,α-trehalose, α-maltosyl α,α-trehalose, α-maltotriosyl α,α-trehalose, and α-maltotetraosyl α,α-trehalose. In one embodiment, the glycosylated derivative of trehalose contains α-glucosyl α,α-trehalose in an amount of about 5 mass % or more, about 10 mass % or more, or about 30 mass % or more in terms of anhydride with respect to the total amount of carbohydrates. For example, a carbohydrate containing a glycosylated derivative of α,α-trehalose (product name: “Hallodex”; available from Hayashibara Co., Ltd.) and a carbohydrate obtained by hydrogenating the above-mentioned carbohydrate to convert coexisting carbohydrates into sugar alcohols thereof (product name: “Tornare”; available from Hayashibara Biochemical Laboratories, Inc.; containing the glycosylated derivative of α,α-trehalose in an amount of 47% and water in an amount of 26%) are commercially available as the glycosylated derivatives of trehalose.

Examples of the acylated derivatives include acetylated trehalose, and trehalose acylated so as to be provided with a long-chain acyl group (that has 12 or more carbon atoms and may have 24 or less carbon atoms, for example). Examples of the pharmaceutically acceptable salts of sulfated trehalose and phosphorylated trehalose include alkali metal salts such as sodium salts and potassium salts; alkali earth metal salts such as calcium salts and magnesium salts; organic amine salts such as ammonium salts, triethanolamine salts, and. triethylamine salts; and salts of basic amino acids such as lysine salts and arginine salts.

The pharmaceutical composition according to one embodiment of the present invention can contain trehalose or a trehalose derivative as an active ingredient. The pharmaceutical composition according to one embodiment of the present invention may contain only trehalose or a trehalose derivative as an active ingredient, or may further contain another active ingredient. On the other hand, the pharmaceutical composition according to one embodiment may be used together with another active ingredient instead of containing another active ingredient.

Examples of another active ingredient include one or more of steroid drugs such as inhaled steroid drugs, broncho dilators, antibacterial drugs, antivirus drugs, cytokine inhibitors, leukotriene receptor antagonists, mast cell degranulation inhibitors, antiallergic agents, methylxanthine-based drugs, anticholinergic agents, antihistamine agents, airway secretion promoters, airway lubricants, and airway mucosa repairing agents. Further examples of another active ingredient include vitamin D, which is reported to reduce the death rate of Covid-19.

Examples of the steroid drugs include clobetasol propionate, diflorasone diacetate, fluocinonide, mometasone furoate, betamethasone dipropionate, betamethasone butyrate propionate, betamethasone valerate, difluprednate, budesonide, diflucortolone valerate, amcinonide, halcinonide, dexamethasone, dexamethasone propionate, dexamethasone valerate, dexamethasone acetate, hydrocortisone acetate, hydrocortisone butyrate, hydrocortisone butyrate propionate, deprodone propionate, prednisolone valerate acetate, fluocinolone acetonide, beclometasone dipropionate, triamcinolone acetonide, flumethasone pivalate, alclometasone propionate, clobetasone butyrate, prednisolone, beclomethasone propionate, fludroxycortide, cortisone acetate, hydrocortisone, sodium hydrocortisone phosphate, sodium hydrocortisone succinate, fludrocortisone acetate, prednisolone acetate, sodium prednisolone succinate, prednisolone butylacetate, sodium prednisolone phosphate, halopredone acetate, methylprednisolone, methylprednisolone acetate, sodium methylprednisolone succinate, triamcinolon, triamcinolon acetate, sodium dexamethasone phosphate, dexamethasone palmitate, paramethasone acetate, betamethasone, fluticasone propionate, flunisolide, ST-126P, ciclesonide, dexamethasone palmithionate, mometasone furancarbonate, prasteron sulfonate, deflazacort, methylprednisolone suleptanate, sodium methylprednisolone succinate, and ciclesonide. Out of these compounds, examples of steroids include dexamethasone, hydrocortisone, prednisolone, and ciclesonide.

Examples of the broncho dilators include short-acting inhalants and long-acting inhalants. Examples of the short-acting inhalants include salbutamol, procaterol, and fenoterol. Examples of the long-acting inhalants include salmeterol and formoterol budesonide. When the pharmaceutical composition according to one embodiment is used together with a broncho dilator, the broncho dilator may be a plaster such as tulobuterol or an internal medicine such as formoterol.

Examples of the antibacterial drugs include antibiotics such as cefuroxime sodium, meropenem trihydrate, netilmicin sulfate, sisomicin sulfate, ceftibuten, tobramycin, doxorubicin, astromicin sulfate, and cefetamet pivoxil hydrochloride.

Examples of the antivirus drugs include oseltamivir, zanamivir, inavir, peramivir, baloxavir, remdesivir, favipiravir, lopinavir, and ritonavir. Other examples of the antivirus agents include compounds such as chloroquine, hydroxychloroquine, and nafamostat that have antiviral activity against specific viruses including a virus that causes Covid-19.

Examples of the cytokine inhibitors include interleukin inhibitors such as tocilizumab and sarilumab, and cytokine signaling pathway inhibitors such as ruxolitinib.

Examples of the leukotriene receptor antagonists include pranlukast and montelukast.

An example of the mast cell degranulation inhibitors is a cromoglycate inhalant.

Examples of the antiallergic agents include suplatamide, ketotifen, and azelastine.

Examples of the methykanthine-based drugs include theophylline and aminophylline.

Examples of the anticholinergic agents include ipratropium bromide and oxitropium.

Examples of the antihistamine agents include diphenhydramin, chlorpheniramine, promethazine, hydroxyzine, cyproheptadine, epinastine, cetirizine, loratadine, fexofenadine, and bilastine.

An example of the airway secretion promoters is bromhexine hydrochloride.

An example of the airway lubricants is ambroxol.

An example of the airway mucosa repairing agents is carbocysteine.

The pharmaceutical composition according to one embodiment of the present invention may contain an additive in addition to the active ingredient. Examples of the additive include excipients, stabilizers, preservatives, buffers, corrigents, suspending agents, emulsifiers, flavoring agents, solubilizing agents, coloring agents, thickening agents, and tonicity agents. The pharmaceutical composition according to one embodiment contains, as flavoring agents, one or more of camphor, menthol, borneol, and other terpenoid compounds.

There is no particular limitation on the medicinal substance form of the pharmaceutical composition according to one embodiment of the present invention. Examples of the medicinal substance form of the pharmaceutical composition according to one embodiment include a tablet, a pill, powdered medicine, granular medicine, liquid medicine, a suspension, syrup, a capsule, an injection, an ointment, a cream, and a lotion. Also, there is no particular limitation on the administration route of the pharmaceutical composition according to one embodiment of the present invention. For example, the pharmaceutical composition according to one embodiment may be parenterally administered through intravenous administration, subcutaneous administration, intramuscular administration, dermal administration, or nasal administration, or may be administered by inhalation.

On the other hand, when a pharmaceutical composition is used in accordance with the application purpose described later, the pharmaceutical composition according to one embodiment of the present invention can be administered by inhalation from the viewpoint of deliver the pharmaceutical composition directly to the lung. An administration device such as a spray, nebulizer, atomizer, or inhaler can be used for inhalation.

The pharmaceutical composition according to one embodiment is administered using an atomization device such as a nebulizer. In this case, the pharmaceutical composition may also be a liquid formulation. For example, the pharmaceutical composition according to one embodiment may contain a solvent such as water in addition to trehalose or a trehalose derivative.

The pharmaceutical composition according to one embodiment is administered using a powder inhaler such as a dry powder inhaler (DPI). In this case, the pharmaceutical composition may also be a powder formulation. For example, the pharmaceutical composition according to one embodiment may contain a carrier such as lactose in addition to trehalose or a trehalose derivative.

The pharmaceutical composition according to one embodiment is administered using an aerosol inhaler such as a pressurized metered dose inhaler (pMIDI) or a breath activated inhaler (BM). In this case, the pharmaceutical composition may be a liquid formulation. For example, the pharmaceutical composition according to one embodiment may contain a solvent such as ethanol in addition to trehalose or a trehalose derivative. In this case, an aerosol propellant such as an alternative for chlorofluorocarbon can be used to deliver the pharmaceutical composition to a disease site.

The pharmaceutical composition according to one embodiment is administered using an injection device such as a spray or an atomizer. In this case, the pharmaceutical composition may be a liquid formulation. For example, the pharmaceutical composition according to one embodiment may contain a solvent such as water in addition to trehalose or a trehalose derivative.

The pharmaceutical composition according to one embodiment is a liquid formulation to be intravenously administered. The pharmaceutical composition according to one embodiment can be administered through an intravascular catheter from the viewpoint of improving the pulmonary delivery efficiency of the pharmaceutical composition. In particular, the pharmaceutical composition can be administered to the central vein through a catheter indwelling near the right atrium. For example, administering the pharmaceutical composition through a deep central venous catheter makes it possible to allow trehalose or a trehalose derivative to reach the blood vessels in the alveoli via the pulmonary artery and to be diffused in the alveoli. In this case, the pharmaceutical composition according to one embodiment may be in the form of an infusion containing trehalose or a trehalose derivative.

The amount of trehalose or a trehalose derivative contained in the pharmaceutical composition according to one embodiment of the present invention can be selected as appropriate in accordance with the administration route, the application purpose, and the like. The pharmaceutical composition according to one embodiment may contain trehalose or a trehalose derivative in an amount of 1 wt % or more or 4 wt % or more, and may contain trehalose or a trehalose derivative in an amount of 20 wt % or less or 12 wt % or less. For example, the pharmaceutical composition according to one embodiment is a liquid. formulation containing trehalose or a trehalose derivative in an amount of 4 to 12 wt % and a solvent. The pharmaceutical composition according to another embodiment may contain trehalose or a trehalose derivative in an amount of 3.5 wt % or more or 3.75 wt % or more. The pharmaceutical composition containing trehalose or a trehalose derivative in an amount of 3.5 wt % or more or 4 wt % or more has high ability to form a trehalose layer covering a cell membrane or a virus, and can thus be used to strengthen the barrier function of a cell membrane or suppress the entry of a virus into a cell. Accordingly, such a liquid formulation is suitable for nasal administration using a spray or nebulizer. Also, the pharmaceutical composition according to one embodiment to be administered to the central vein may contain trehalose or a trehalose derivative at a high concentration. For example, the pharmaceutical composition according to one embodiment may contain trehalose or a trehalose derivative in an amount of 10 wt % or more or 20 wt % or more, and may contain trehalose or a trehalose derivative in an amount of 50 wt % or less or 40 wt % or less. Such a liquid formulation can be administered through a central venous catheter.

In an embodiment, the above described liquid formulation may have a pH of 5.1 or above, or 5.6 or above, and may have a pH of 6.2 or below, or 6.1 or below. Such a liquid formulation is suitable for protect a respiratory tract or a lung from damage caused by infection with coronaviruses. Namely, the S protein (spike protein) on the surface of the coronavirus is a basic protein and has an isoelectric point of 8.91. The S protein has a higher activity when the pH is around this isoelectric point. On the other hand, an immunoglobulin IgA is an acidic protein and has an isoelectric point of 5.05. This IgA has a higher activity when the pH is around this isoelectric point. Therefore, with the administration of the liquid formulation with the above pH, the activity of the S protein is reduced while the IgA has high activity, thereby the antibody reaction is expected to be enhanced. Furthermore, with the administration of the liquid formulation with the above pH, the S protein will have a positive charge while the IgA will have a negative charge, thereby the antibody reaction is expected to be accelerated through electrostatic interaction between the S protein and the IgA. In addition, a charge on a surface of mucosa, which is shifted toward negative due to virus infection, shifts toward positive with the liquid formulation, and therefore there will be repulsion between the mucosa and the S protein while there will be attraction between the mucosa and the IgA, thereby increase of IgA concentration on the surface is the mucosa is expected. Such a liquid formulation can be administered to the respiratory tract as mist through a spray or a nebulizer.

The pharmaceutical composition according to one embodiment of the present invention is used such that an effective dose of trehalose or a trehalose derivative is administered to a patient. The effective dose of trehalose or a trehalose derivative varies depending on the administration route, the age of a patient, the sex, and the degree of the disease, and is 0.01 g/day or more or 0.1 g/day or more, for example, and 10 g/day or less or 1 g/day or less, for example. The administration frequency is 1 to 3 times/day, or may be every 3 to 5 hours, for example, but is not limited thereto. The formulation can be prepared such that these conditions are satisfied.

Hereinafter, the application purpose of the pharmaceutical composition according to one embodiment of the present invention will be described. That is, a method according to one embodiment of the present invention includes administering an effective amount of trehalose or a trehalose derivative to a patient for at least one of the following purposes.

The pharmaceutical composition according to this embodiment can be used to protect the lung from oxygen. Oxygen inhalation is often administered to a patient such as a patient with an infectious pulmonary disease, and a high concentration of oxygen is administered in this case. Also, in a case of using a ventilator, a high concentration of oxygen is administered to a patient. However, oxygen free radicals are likely to be generated during the administration of oxygen, and oxygen free radicals may directly cause cell damage in the lung. Moreover, oxygen free radicals may cause damage to the trachea, vascular endothelial cells, alveolar epithelial cells, and the like via inflammatory cells such as macrophages. As a result, these may lead to atelectasis, a pulmonary edema, alveolar hemorrhage, a decrease in a pulmonary surfactant, fibrin deposition, thickening of the alveolar septa, a decrease in pulmonary compliance, a decrease in diffusing capacity, and an increase in A-aDO₂. In general, as the limit of the administration of a high concentration of oxygen, it is recommended to set the oxygen administration period to 6 hours or shorter when the oxygen concentration is 100%, to 12 hours or shorter when the oxygen concentration is 80%, and to 48 hours or shorter when the oxygen concentration is 50%. In addition to such oxidative stress, oxidative stress due to viral infection also causes interstitial pneumonia.

On the other hand, as shown in Example 6, the pharmaceutical composition according to this embodiment can protect cells such as pulmonary bronchial epithelial cells or alveolar epithelial cells from oxygen free radicals. Accordingly, the pharmaceutical composition according to this embodiment can be administered to a patient in order to protect the lung from oxygen particularly when oxygen inhalation is administered or a ventilator is used. For example, when the condition of pneumonia has worsened to dyspnea and thus a high concentration of oxygen is supplied using an oxygen inhalation mask or a ventilator, the pharmaceutical composition according to this embodiment can suppress the progress of pulmonary tissue lesion, particularly interstitial pneumonia, caused by oxidative stress from a high concentration of oxygen. For example, the pharmaceutical composition according to this embodiment may be administered to a patient together with oxygen during oxygen inhalation, or may be mixed with oxygen and supplied to a patient.

Moreover, the pharmaceutical composition according to this embodiment can be used to protect the lung from dryness. Dry gas is fed to the upper respiratory tract when oxygen inhalation is administered or a ventilator is used. Accordingly, water is also depleted from the respiratory tract, which leads to dryness of or cell damage in the airway mucosa, a decrease in ciliary movement, dryness or solidification of sputum, tube occlusion by sputum, and the like. As a result, the pulmonary cells may be damaged. Infectious pulmonary damage is often developed under a low-temperature dry atmosphere in winter, and in particular, drying stimulus is likely to be applied to the lung in such an environment.

On the other hand, as shown in Example 8, the pharmaceutical composition according to this embodiment can protect cells such as the pulmonary bronchial epithelial cells or alveolar epithelial cells from drying stress caused by drying stimulus. Accordingly, the pharmaceutical composition according to this embodiment can be administered to a patient in order to protect the lung from dryness particularly when oxygen inhalation or a ventilator is used. For example, when high-concentration oxygen inhalation or a ventilator is used for a patient with an infectious pulmonary disease, the pharmaceutical composition according to this embodiment can suppress the progress of pulmonary tissue lesion. Moreover, trehalose has a moisturizing effect and a cytoprotective function, and therefore, administering the pharmaceutical composition according to this embodiment using a nebulizer makes it possible to suppress cell death (cellular lesion) and prevent sputum from being dried to have reduced viscosity and facilitate the expectoration.

Moreover, the pharmaceutical composition according to this embodiment can protect the lung from pressure damage induced by a ventilator. When positive pressure ventilation is performed using a ventilator, high expansion pressure is applied to the lung. In particular, when the condition of pneumonia has worsened, spaces in the respiratory tract and alveoli are reduced, and therefore, higher pressure is applied from the ventilator in order to increase the spaces. At this time, pulmonary mucosa is damaged due to the expansion pressure, which causes interstitial pneumonia.

As shown in Examples 6 and 8, the pharmaceutical composition according to this embodiment can protect cells from stimulus. Accordingly, the pharmaceutical composition according to this embodiment can protect the lung from pressure damage induced by a ventilator. It is considered that the mechanism by which the pharmaceutical composition according to this embodiment protects the lung from pressure damage induced by a ventilator is as follows: since trehalose increases the strength of intercellular phospholipid bonding (C. S. Pereira et al. Biophysical Journal 2008, 95, 3525-3534.), the gaps are not formed between cells even when expansion pressure is applied, and thus the onset of interstitial pneumonia is suppressed. The pharmaceutical composition according to this embodiment can be administered as an inhalant from the viewpoint of delivering trehalose directly to the pulmonary bronchial epithelial cells or alveolar epithelial cells and thus increasing the strength of the cells.

Moreover, the pharmaceutical composition according to this embodiment can suppress the propagation of cell death to surrounding cells induced by cell death. In the case of pulmonary tissue lesion caused by an infectious pulmonary disease and the like, accidental cell death (necrosis) occurs as a result of rupture of cells and leakage of the contents thereof. In necrosis, the whole cell and mitochondria gradually swell, and the cytoplasm also changes. Ultimately, the cell membrane ruptures, which leads to cytolysis involving inflammation. As a result, even normal surrounding cells are also injured.

As shown in Example 7, the pharmaceutical composition according to this embodiment has an action of protecting cells from the propagation of cell death to surrounding cells, which is induced by cell disruption caused by damage or heat shock. Accordingly, the pharmaceutical composition according to this embodiment can be used to suppress the propagation of cell death to surrounding cells induced by cell death. For example, the pharmaceutical composition according to this embodiment can suppress the propagation of cell death to surrounding cells caused by cell contents (mainly lysosomes) resulting from cell death induced by direct damage by bacteria or viruses. In particular, the pharmaceutical composition according to this embodiment can be used to suppress an increase in the severity of a disease, particularly viral interstitial pneumonia, caused by the propagation of cell death.

Moreover, the pharmaceutical composition according to this embodiment can suppress cell death. It is reported that excessive apoptosis of alveolar epithelial cells is particularly related to the condition of idiopathic interstitial pneumonia. Furthermore, it is conceivable that apoptosis may be closely related to acute inflammation. Inflammation and apoptosis are similar to each other in the sense that originally autologous substances are heterogenized and eliminated, and the molecular mechanisms thereof are very similar to each other. In the inflammatory process, apoptosis occurs in both cells to be eliminated and inflammatory cells, and apoptosis may also promote inflammation. Close interaction between inflammation and apoptosis is a conceivable cause of pulmonary lesion.

As shown in Examples 1, 5, and 6 to 8, the pharmaceutical composition according to this embodiment has a cytoprotective action. In addition, trehalose has an autophagic effect, and therefore, it is possible to keep the homeostasis, which is closely related to apoptosis and acute inflammation, in normal balance (TH1/Th2), and suppress the onset of sepsis caused by a chain of cell death. As described above, the pharmaceutical composition according to this embodiment can be used to promote autophagy in cells to reduce the probability of the occurrence of cell death and thereby suppress cell death.

Moreover, as shown in Examples 6 to 8, the pharmaceutical composition according to this embodiment can protect the pulmonary microvascular endothelial cells or alveolar epithelial cells. That is, the pharmaceutical composition according to this embodiment can protect cells from mechanical stimulus such as dryness or pressure, or chemical stimulus caused by substances resulting from cell death. For example, the pharmaceutical composition according to this embodiment can protect the pulmonary microvascular endothelial cells and alveolar epithelial cells, and prevent necrosis (cell death) induced by direct damage caused by bacteria or viruses and a chain of cell death. Furthermore, as shown in Examples 1 to 5, the pharmaceutical composition according to this embodiment can suppress the production of IgE antibodies from b cells to suppress an increase in the severity of an inflammatory pulmonary disease caused by cytokine storm, which is an overreaction of the immune system caused by chemical mediators from mast cells. Accordingly, the pharmaceutical composition according to this embodiment can also be used to suppress the progress of an infectious pulmonary damage, particularly viral interstitial pneumonia. Moreover, the pharmaceutical composition according to this embodiment is particularly effective for patients with viral pneumonia who undergo oxygen inhalation or use a ventilator.

Moreover, the pharmaceutical composition according to this embodiment can reduce the airway resistance to alleviate anoxic conditions in the lung. When the condition of pneumonia has worsened as mentioned above, the respiratory tract gets narrow, and thus the lung is likely to become hypoxemia. On the other hand, as shown in Example 2, the pharmaceutical composition according to this embodiment has an action of reducing the airway resistance. That is, the pharmaceutical composition according to this embodiment can reduce airway mucosal edema or secretion (sputum) from the respiratory tract to suppress airway occlusion. As described above, the pharmaceutical composition according to this embodiment can prevent airway stenosis caused by infectious pulmonary damage to reduce the airway resistance and to prevent a decrease in respiratory compliance, and can thus alleviate anoxic conditions. In particular, the pharmaceutical composition according to this embodiment can be used to alleviate anoxic conditions in the lung particularly when oxygen inhalation is administered or a ventilator is used. Administering the pharmaceutical composition according to this embodiment also makes it possible to use a ventilator at lower pressure.

Moreover, the pharmaceutical composition according to this embodiment can suppress infection with viruses or bacteria that cause pneumonia. Examples 6 and 8 show that the pharmaceutical composition according to this embodiment has an action of strengthening the barrier function of the cell membrane and. protecting the cell from stimulus. In particular, the host cells include phospholipids. Moreover, the surfaces of the cells are also constituted by phospholipids, and the surfaces of enveloped viruses are also constituted by phospholipids. Trehalose binds to the phospholipid membrane to form a hydrated region, and this hydrated region serves as a barrier (A. Abazari et al. Biophysical Journal, 2014, 107, 2253-2262). As described above, administering trehalose makes it possible to prevent contact between host cells, and viruses and bacteria. and thus suppress infection. In addition, trehalose has a chemical chaperon function, namely a fluidization action exhibited by stabilizing the shapes and structures of cells, and thus viruses or bacteria and the host cell membranes are kept away from each other. Accordingly, the proliferation of viruses or bacteria is suppressed while the antibody productivity per cell is increased.

Moreover, the pharmaceutical composition according to this embodiment can suppress the propagation of infection with viruses or bacteria that cause pneumonia, in the pulmonary bronchial epithelial cells or alveolar epithelial cells. Examples 6 and 8 show that the pharmaceutical composition according to this embodiment has an action of strengthening the barrier function of the cell membrane and protecting the cell from stimulus. Furthermore, phospholipids in the cell membrane bind to one another via hydrogen bonds produced by water, but hydrogen bonds produced by trehalose are stronger than those produced by water, and therefore, administering trehalose makes it possible to improve the mechanical strength of the cell membrane (C. S. Pereira et al. Biophysical Journal 2008, 95, 3525-3534.). Accordingly, due to the administration of trehalose, a greater amount of energy is required for viruses to enter host cells and break out therefrom, and thus the speed of the propagation of viruses among the cells is reduced. Similarly, the speed of cell destruction by bacteria and the speed of the proliferation of bacteria are also reduced.

More specifically, when viruses attach to and enter cells such as mucosal cells of the respiratory tract, hydrophobic interaction between the surfaces of the viruses and the surfaces of the cell membranes plays an important role. On the other hand, trehalose can interact with phospholipids and other molecules on the surfaces of the cell membranes to replace water molecules near the surfaces of the cell membranes and form clustered layers with a thickness of approximately 15 nm that cover the surfaces of the cell membranes. Such properties of trehalose are effective in protecting the structures of the cell membranes under stress. Similarly, trehalose can form clustered layers with a thickness of approximately 15 nm that cover the surfaces of viruses. These actions of trehalose make it possible to inhibit hydrophobic interaction between the surfaces of viruses and the surfaces of cell membranes and suppress the entry of the viruses into the cells.

Moreover, trehalose also has an action of stabilizing the structure of a protein. The reason for this is that trehalose that has attached to proteins and covered them can cause release of hydration water to form glassy matrices, and these matrices can physically protect the proteins and cells including the proteins from abiotic stress. Another reason is that water molecules are thrust away from the vicinity of the proteins by the administration of trehalose, and the hydration radii of the proteins are thus reduced, thus making it possible to reduce the sizes of the proteins and improve the stability thereof. Yet another reason is that trehalose forms hydrogen bonds and thus replace water around the proteins, thus making it possible to maintain the tertiary structures of the proteins and stabilize the proteins. Moreover, trehalose is likely to cover a protein through aggregation thereof on the surface of the protein. Due to these actions, trehalose can inhibit RNA synthesis of viruses by attaching to and covering ribosomes and stabilizing the ribosomes. In addition, trehalose can suppress entry of viruses such as coronaviruses into cells by attaching to and covering spike proteins on the surfaces of the viruses and stabilizing the spike proteins.

As described above, the pharmaceutical composition according to one embodiment is a pharmaceutical composition for protecting the respiratory tract or lung from damage that contains trehalose or a trehalose derivative. Such a pharmaceutical composition can be used to protect the cells of the upper respiratory tract and the cells of the lower respiratory tract including the lung from damage. This damage may be damage caused by viral infection, or damage caused by infection with coronaviruses, such as SARS, VIERS, and Covid-19. Moreover, this damage may be damage caused by oxygen, dryness, or pressure damage. Furthermore, this damage may be damage caused by viruses, bacteria, pollen, house dust, or soot dust. Furthermore, this damage may be damage caused by cell death of surrounding cells, and the cell death of the surrounding cells may be caused by viral infection, oxygen, dryness, or pressure damage.

Furthermore, the pharmaceutical composition according to one embodiment can be administered to a patient suffering from allergic rhinitis, since trehalose can suppress IgE antibody production, inhibit inflammatory cell infiltration, and inhibit leukotriene and histaine, in addition to protecting cell membranes of cells such as mucosal cells and cilia cells. For example, a nasal spray containing trehalose could reduce Total Nasal Allergic Symptom Score (TNSS), i.e., could alleviate nasal itching, rhinorrhea, nasal congestion, and sneezing. The nasal spray containing trehalose could reduce TNSS by more than six points, which was significantly effective compared to a fluticasone nasal spray and a mometasone nasal spray. The trehalose solution composition administered to the nasal cavity and nasal pharynx washes viruses and allergens in the upper respiratory tract, passes through the occipital region of the pharynx, and is swallowed into the esophagus and discharged into the stomach. Excess trehalose solution composition dissolves gel-like nasal mucus matrix and is directly expelled from the nose as a nasal colloid.

### EXAMPLES

### Example 1

Asthma model mice were produced using BALB/c mice (8 to 10 weeks old) and ovalbumin (OVA) serving as an antigen, and examinations were conducted by administering trehalose by inhalation. On the first day and the fourteenth day, a physiological saline solution or OVA was intraperitoneally administered. 20 micrograms of OVA was administered to one mouse using aluminum hydroxide as an adjuvant. From the twenty-seventh day to the thirty-first day, 10% trehalose or distilled water was administered by inhalation for 20 minutes every day. Moreover, from the twenty-eighth day to the thirtieth day, a physiological saline solution or 1% OVA was administered by inhalation for 20 minutes, 1 to 1.5 hours after the above-mentioned inhalation. On the thirty-second day, evaluation was conducted using an assay system for evaluating allergic airway inflammation and hyperresponsive airway.

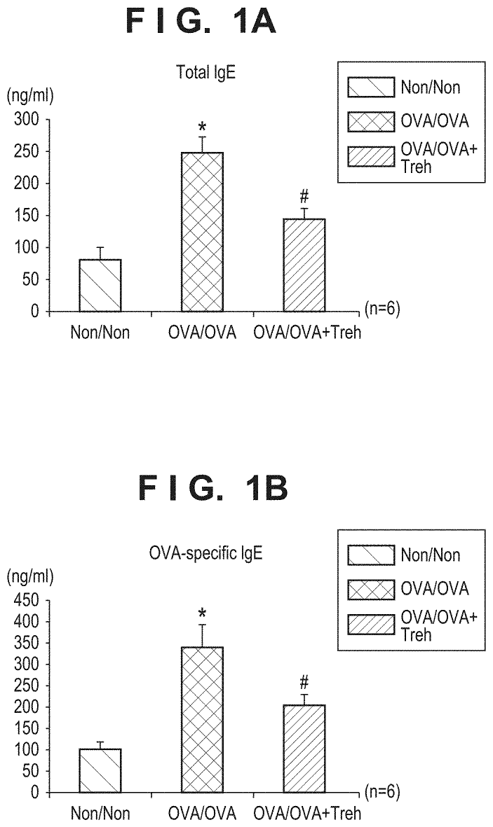

FIGS. 1A and 1B show the measurement results of the IgE antibodylevels in the serum. FIG. 1A shows the total IgE levels, and FIG. 1B shows the levels of IgE specific to the antigen (ovalbumin). It should be noted that, in FIGS. 1A to 5C, * indicates that the measurement results from the OVA-administrated mice (OVA/OVA) were significantly different (P<0.05) from those from non-administrated mice (Non/Non). Moreover, # indicates that the measurement results from the trehalose-OVA-administration mice (OVA/OVA+Treh) were significantly different (P<0.05) from those from distilled water-OVA-administration mice (OVA/OVA). FIGS. 1A and 1B show that trehalose could significantly reduce the production amount of IgE. Suppressing the production of IgE antibodies makes it possible to suppress an increase in the severity of pulmonary tissue lesion caused by cytokine storm, which is an overreaction of the immune system caused by chemical mediators from mast cells. Accordingly, it can be said that trehalose is effective in protecting the pulmonary microvascular endothelial cells or alveolar epithelial cells particularly in infectious pulmonary damage.

### Example 2

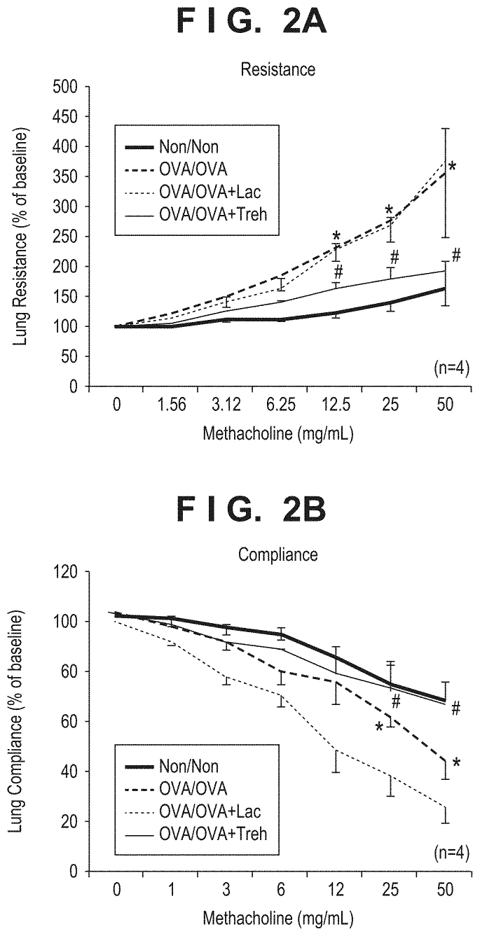

The airway resistance and the compliance of the model mice of Example 1 were measured. FIG. 2A shows the relationships between the amounts of a muscarine receptor stimulant and the airway resistance, which were measured in Example 2. FIG. 2B shows the relationships between the amounts of a muscarine receptor stimulant and the compliance. FIGS. 2A and 2B show that trehalose significantly reduced the airway resistance, and significantly increased the compliance. These results mean that reducing the airway resistance by using trehalose makes it possible to alleviate anoxic conditions in the lung. On the other hand, FIGS. 2A and 2B also show the results of the experiment in which lactose was used instead of trehalose (OVA/OVA+Lac). As shown in FIGS. 2A and 2B, OVA/OVA and OVA/OVA+Lac were not different from each other in the airway resistance and the compliance. This shows that lactose does not have an action of suppressing an increase in the airway resistance. Moreover, it was found from the comparison of OVA/OVA+Treh and OVA/OVA+Lac that the action of suppressing an increase in the airway resistance is significantly higher in trehalose than in lactose.

### Example 3

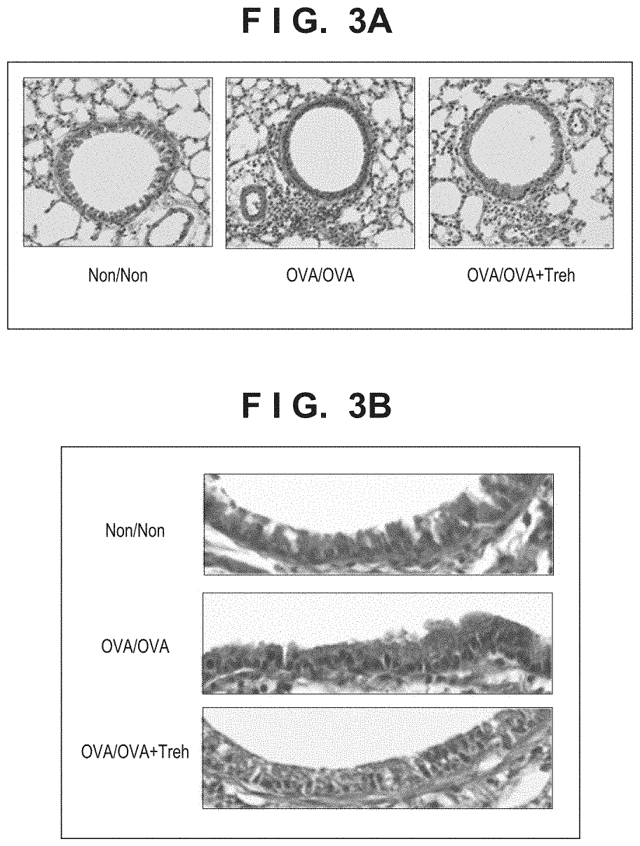

Pulmonary tissue pathological sections were produced from the model mice of Example 1, and the diseases state was observed through hematoxylin-eosin (H.E.) staining and periodic acid-Schiff (PAS) staining. FIGS. 3A and 3B show the results of H.E. staining and the results of PAS staining, respectively. As shown in FIGS. 3A and 3B, it was observed that primary inhalation of trehalose made it possible to suppress damage to the alveolar epithelial cells and protect the cells. It was observed that trehalose could particularly suppress damage to the alveolar epithelial cells (airway inflammatory cell infiltration or airway epithelial cell goblet cell hyperplasia) that is mainly caused by eosinophils and the hypersensitivity of the respiratory tract.

### Example 4

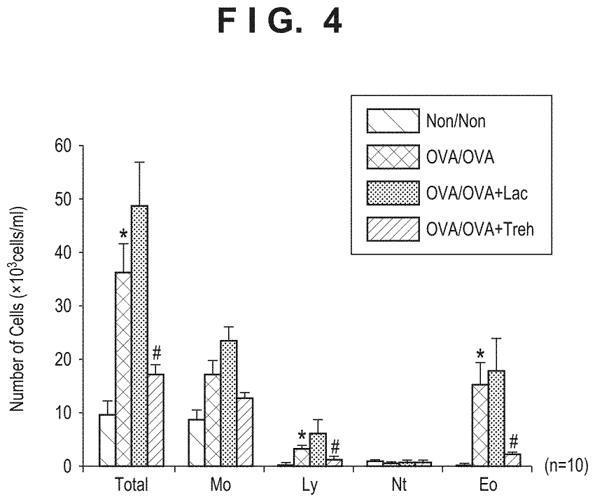

Bronchoalveolar lavage was performed on the model mice of Example 1, and the numbers of various cells obtained through the bronchoalveolar lavage were compared and examined. It should be noted that, in this example, 0.5 mL of a physiological saline solution containing 50 μg of ovalbumin (OVA) and 1 mg of aluminum hydroxide gel serving as an adjuvant was administered to OVA-administration mice (OVA/OVA) every 12 days. FIG. 4 shows the measurement results of the numbers of inflammation-inducing cells contained in the bronchoalveolar lavage fluids in Example 4. In FIG. 4, “Total” indicates a total of cells, “Mo” indicates monocytes, “Ly” indicates lymphocytes, “Eo” indicates eosinophils, and “Nt” indicates neutrophils. FIG. 4 shows that trehalose significantly reduced cell release. As described above, it was found that the release of inflammation-inducing cells from the bronchoalveoli was suppressed by the administration of trehalose. Accordingly, trehalose can suppress pulmonary tissue lesion by suppressing the release of inflammation-inducing cells from the bronchoalveoli. On the other hand, FIG. 4 also shows the results of the experiment in which lactose was used instead of trehalose (OVA/OVA±Lac). As shown in FIG. 4, it was found that lactose does not have an action of suppressing the release of inflammation-inducing cells, and the action of suppressing the release of inflammation-inducing cells is significantly higher in trehalose than in lactose.

### Example 5

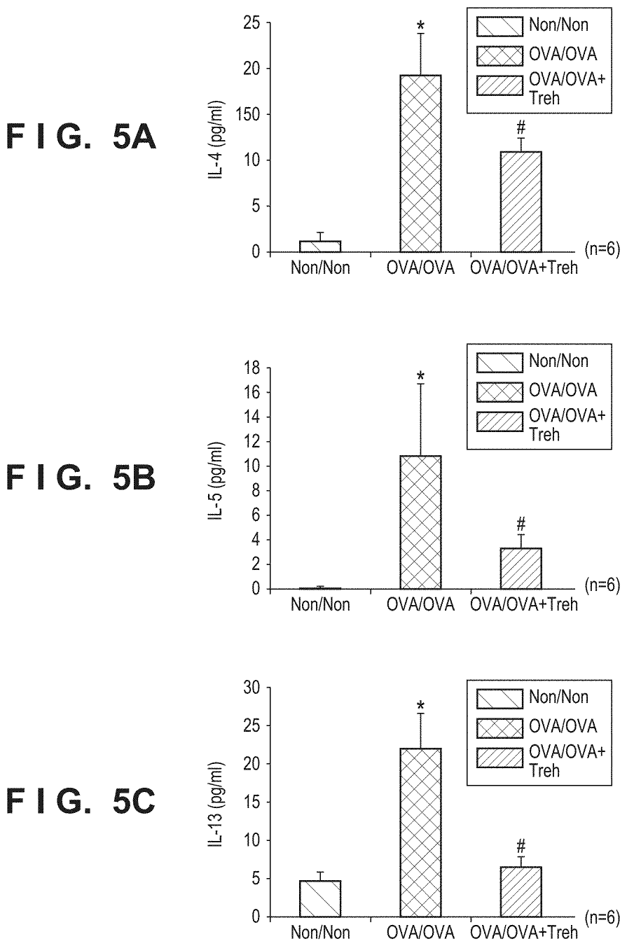

The cytokine levels in the bronchoalveolar lavage fluids obtained in Example 4 were measured using an ELISA measurement kit. FIGS. 5A to 5C show the measurement results. FIG. 5A shows the IL-4 levels, FIG. 5B shows the IL-5 levels, and FIG. 5C shows the IL-13 levels. It should be noted that interferon (IFN-γ) was not detected from any of the groups. FIGS. 5A to 5C show that trehalose can significantly suppress the expression of cytokines. Accordingly, trehalose can suppress pulmonary tissue lesion by suppressing the expression of cytokines.

### Example 6

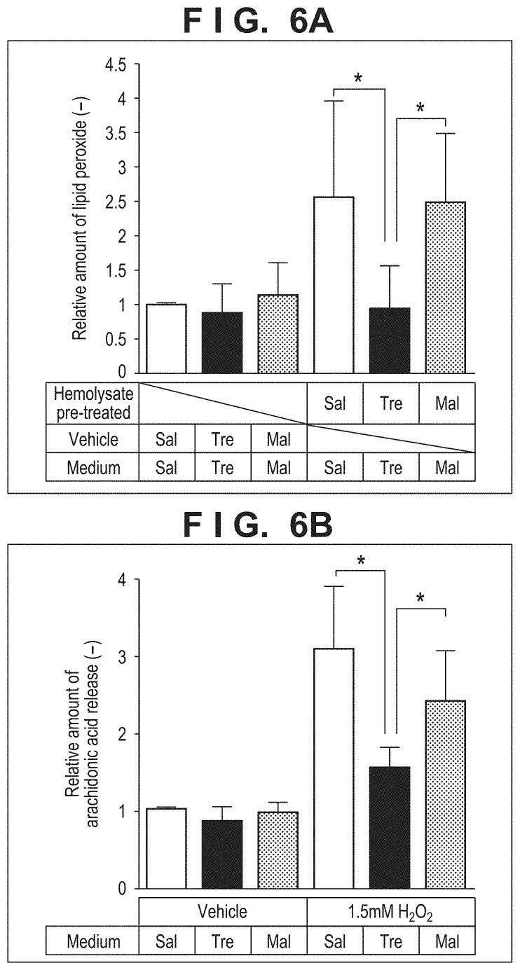

Suppression of PLA2 activation caused by applying hydrogen peroxide stimulus to RAW264.7 cells (macrophages) was measured using [³H]-AA release assay. FIGS. 6A and 6B show the measurement results showing the influence of trehalose on hemolysate or oxygen free radicals. FIGS. 6A and 6B show the measurement results in the case where a culture medium or hemolysate contains a physiological saline solution (Sal), 5% trehalose (Ire), or 5% maltose (Mal). FIG. 6A shows the measurement results of lipid peroxides (LPO). That is, a low LPO level indicates that cells were protected from hemolysate. In this experiment, RAW264.7 cells were treated with 10% hemolysate for 3 hours. The hemolysate was prepared as a supernatant by suspending erythrocytes of a Wistar rat in a saline solution, an aqueous solution of trehalose, or an aqueous solution of maltose, sonicating the resulting suspension, and then centrifuging the suspension. The LPO level was measured through a colorimetric analysis using a divalent iron molecule. FIG. 6B shows the effect of trehalose on arachidonic acid in cells treated with hydrogen peroxide. In this experiment, RAW264.7 cells were labeled with [3H] arachidonic acid overnight and then treated with hydrogen peroxide for 3 hours. The level of released arachidonic acid was quantified by measuring the radioactivity of the collected culture medium. It is known that hydrogen peroxide stimulus induces the release of arachidonic acid via peroxidation of lipids, and therefore, a low level of released arachidonic acid indicates that cells are protected from oxygen free radicals. In FIGS. 6A and 6B, * indicates that there is a significant difference (P<0.05).

It was found from FIGS. 6A and 6B that trehalose has an action of protecting cells from oxygen free radicals and hemolysate. Accordingly, it is shown that trehalose can suppress pulmonary tissue lesion caused by oxygen.

### Example 7

It was examined if trehalose suppresses the production of cytokine storm induced by cell lysate, which serves as a model of the propagation of cell death.

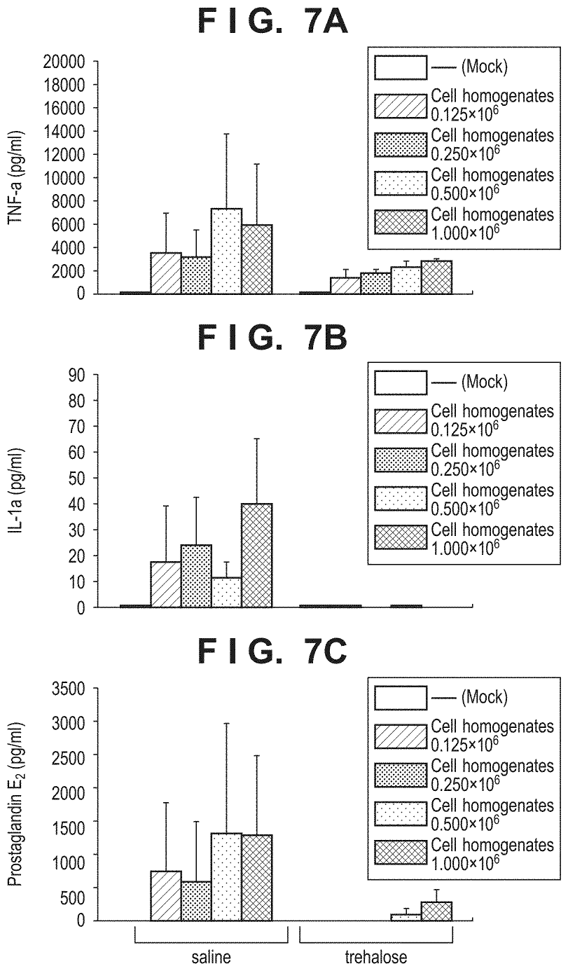

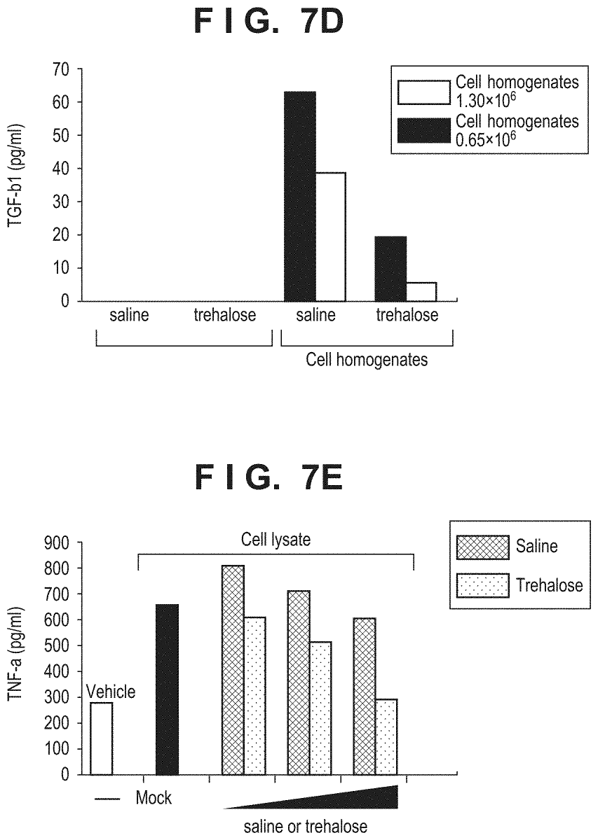

RAW264.7 cells cultured in a 225-cm² flask were homogenized in a physiological saline solution (saline) or a 10% aqueous solution of trehalose (trehalose) using a homogenizer, and cell lysate was thus prepared. The above-mentioned cell lysate was added to RAW264.7 cells that were being cultured in 6 wells, and the cells were cultured for another 8 hours. Then, TNF-α and IL-1α were measured using ELISA (manufactured by R&D Systems), and prostaglandin E₂ was measured using EIA (manufactured by Cayman Chemical). FIGS. 7A, 7B, and 7C show the measurement results of TNF-α, IL-1α, and prostaglandin E₂, respectively.

Moreover, RAW264.7 cells cultured in a 225-cm² flask were homogenized in a physiological saline solution (saline) or a 10% aqueous solution of trehalose (trehalose) using a homogenizer, and cell lysate was thus prepared. The above-mentioned cell lysate was added to RAW264.7 cells that were being cultured in 6 wells, and the cells were cultured for another 8 hours. Then, the culture medium was collected. The culture medium was treated with acid, and then TGF-β1 in the culture medium was measured using ELISA (manufactured by R&D Systems). FIG. 7D shows the measurement results.

In the above-mentioned tests, the cell lysate obtained through homogenization using a homogenizer was used as a model of cell disruption caused by damage (including a surgical operation). As shown in FIGS. 7A to 7D, the levels of cytokines (TNF-α, IL-1α, prostaglandin E₂, and TGF-β1) induced by the cell lysate were significantly reduced by using trehalose. Moreover, it was confirmed that suppression of the TNT-α production depends on the dosage of trehalose. These results indicate that trehalose can protect cells from the propagation of cell death to surrounding cells that is induced by cell disruption caused by damage.

Moreover, RAW264.7 cells cultured in a 225-cm² flask were collected, and heat shock was applied to the cells at 53° C. for 10 minutes. Then, the cells were left to stand at 37° C. for 5 hours to induce cell death, and the resulting solution was used as a cell solution. Thereafter, a physiological saline solution (saline) or a 10% aqueous solution of trehalose (trehalose) was added to the cell solution, and the resulting solution was treated on ice for 30 minutes. The above-mentioned cell solution was added to RAW264.7 cells that were being cultured in 6 wells, and the cells were cultured for another 8 hours. Then, TNF-α was measured using ELISA (manufactured by R&D Systems). FIG. 7E shows the measurement results.

In this test, the cell solution including dead cells induced by heat shock was used as a model of cell disruption caused by a burn. As shown in FIG. 7E, the level of a cytokine (TNF-α) induced by the cell solution was significantly reduced by using trehalose. This result indicates that trehalose can also protect cells from the propagation of cell death to surrounding cells that is induced by cell disruption caused by a burn.

### Example 8

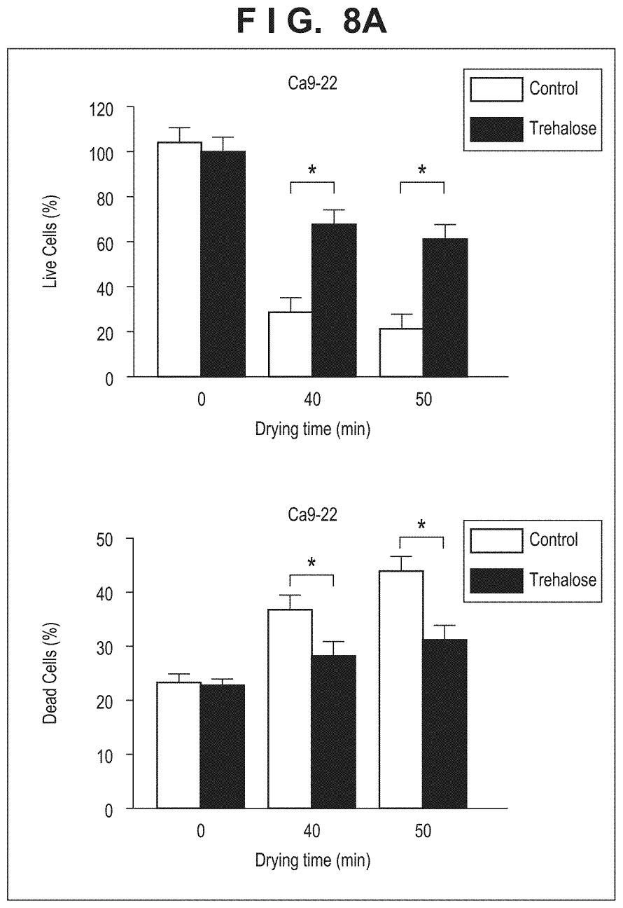

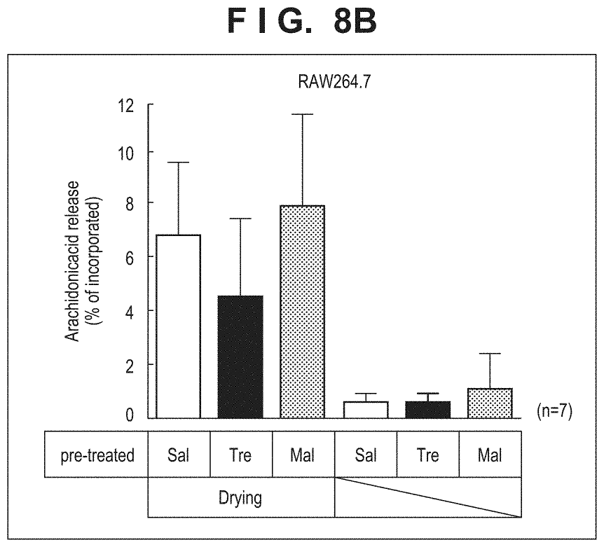

The oral epithelial cell line (Ca9-22) maintained in DMEM-10% FBS was treated with [³H]-arachidonic acid overnight. Pretreatment with a physiological saline solution (Sal), 7.5% trehalose (Tre), or 7.5% maltose (Mal) was conducted for 15 minutes, and then the cells were dried for 7 minutes. Subsequently, a culture medium was added, and then the cells were cultured for 1 hour. FIG. 8A shows the results of LIVE/DEAD assay. FIG. 8B shows the results of assay of the level of released arachidonic acid in which tritium in the culture supernatant was analyzed using a liquid scintillation counter.

FIGS. 8A and 8B show that trehalose suppresses cell death caused by drying stimulus, and suppresses PLA2 activation caused by drying stimulus. These results indicate that trehalose can suppress pulmonary tissue lesion caused by dryness.

The invention is not limited to the foregoing embodiments, and various variations/changes are possible within the spirit of the invention.

1. A method of protecting a respiratory tract or a lung from damage, comprising administering a pharmaceutical composition to a patient in need thereof, wherein the pharmaceutical composition comprises trehalose or a trehalose derivative.

2. The method according to claim 1, wherein the method protects the respiratory tract or the lung from damage caused by infection with coronaviruses.

3. The method according to claim 1, wherein the method protects the respiratory tract or the lung from damage by preventing contact between cells of the respiratory tract or the lung and viruses.

4. The method according to claim 1, wherein the method protects the respiratory tract or the lung from damage caused by cell death of surrounding cells.

5. The method according to claim 1, wherein the method protects the respiratory tract or the lung from damage caused by oxygen, dryness, or pressure damage.

6. The method according to claim 1, wherein the method protects the respiratory tract or the lung from damage by strengthening a barrier function of cell membranes in the respiratory tract or the lung.

7. The method according to claim 1, wherein the method protects the respiratory tract or the lung from damage by binding to cell membranes in the respiratory tract or the lung to form barriers.

8. The method according to claim 1, wherein the method protects the respiratory tract or the lung from damage by forming hydrated regions on surfaces of cells of the respiratory tract or the lung.

9. The method according to claim 1, wherein the method protects the respiratory tract or the lung from damage by stabilizing structures of cells of the respiratory tract or the lung.

10. The method according to claim 1, wherein the method protects the respiratory tract or the lung from damage by stabilizing structures of proteins in cells of the respiratory tract or the lung.

11. A method of protecting a lung from pressure damage induced by a ventilator, comprising administering a pharmaceutical composition to a patient in need thereof, wherein the pharmaceutical composition comprises trehalose or a trehalose derivative.

12. A method of suppressing spread of infection with viruses or bacteria that cause pneumonia in pulmonary bronchial epithelial cells or alveolar epithelial cells, comprising administering a pharmaceutical composition to a patient in need thereof, wherein the pharmaceutical composition comprises trehalose or a trehalose derivative.

13. The method according to claim 1, wherein the pharmaceutical composition is administered to a patient to which a high concentration of oxygen is being administered. The method according to claim 1, wherein the pharmaceutical composition is administered to a patient with infectious pulmonary damage.

15. The method according to claim 1, wherein the pharmaceutical composition further comprises an inhaled steroid and/or a broncho dilator.

16. The method according to claim 1, wherein the pharmaceutical composition comprises an antibacterial drug and/or an antivirus drug.

17. The method according to claim 1, wherein the pharmaceutical composition further comprising a terpenoid compound, camphor, menthol, borneol, an airway secretion promoter, an airway lubricant, and/or an airway mucosa repairing agent.

18. The method according to claim 1, wherein the pharmaceutical composition further comprising vitamin D.

19. The method according to claim 1, wherein the pharmaceutical composition is a liquid formulation containing a trehalose or a trehalose derivative in an amount of 3.5 to 2.0 wt % and a solvent.

20. The method according to claim 1, wherein the pharmaceutical composition is administered to a patient together with oxygen during oxygen inhalation.

21. The method according to claim 1, wherein the pharmaceutical composition is to be atomized and then inhaled by a patient.

22. The method according to claim 1, wherein the pharmaceutical composition is a liquid formulation that is nasally administered using a spray or a nebulizer and contains trehalose or a trehalose derivative in an amount of 3.5 to 20 wt % and a solvent.

23. The method according to claim 19, wherein the pharmaceutical composition is a liquid formulation with a pH of 5.1 or more and 6.2 or less.

24. The method according to claim 1, wherein the pharmaceutical composition is centralvenously administered.

25. The method according to claim 24, wherein the pharmaceutical composition is a liquid formulation that is administered through a central venous catheter.

26. The method according to claim 24, wherein the pharmaceutical composition is a liquid formulation that is administered to a central vein and. contains trehalose or a trehalose derivative in an amount of 10 wt % or more and aqua solution.

27. The method according to claim 1, wherein the pharmaceutical composition is in a form of dry powder and is stored in an inhaler.

28. A nasal spray containing a pharmaceutical composition, wherein the pharmaceutical composition is a liquid formulation that is nasally administered using a spray or a nebulizer and contains trehalose or a trehalose derivative in an amount of 3.5 to 20 wt % and aqua solution.
